(19) United States
(12) Patent Application Publication
(10) Pub. No.: US 2003/0032180 A1
(43) Pub. Date: Feb. 13, 2003

(54) Reconstituted cell lines prepared by nuclear transfer between differentiated cells

(76) Inventor: Larissa V. Strelchenko, DeForest, WI (US)

Correspondence Address: DEWITT ROSS & STEVENS S.C., 8000 EXCELSIOR DR, SUITE 401, MADISON, WI, 53717-1914, US

(21) Appl. No.: 10/226,671

(22) Filed: Aug. 23, 2002

Related U.S. Application Data
(63) Continuation-in-part of application No. 09/628,789, filed on Jul. 29, 2000.

(51) Int. Cl. C12N 5/08; C12N 15/85

(52) U.S. Cl. 435/366; 435/455

(57) ABSTRACT

Disclosed are methods of generating reconstituted cell lines and the cell lines so generated. Specifically described are methods wherein a mammalian, differentiated, diploid donor cell or nucleus is transferred into a mammalian, differentiated, diploid enucleated host cell. The donor cell or nucleus donor nucleus and the host cell can be derived from the same species or different species. The resulting nuclear transfer unit is then incubated to yield a reconstituted mammalian cell line. The cell lines can be generated to express desired human or non-human cell-surface antigens.

### PRIORITY CLAIM

The present application is a continuation-in-part of co-pending application Ser. No. 09/628,789, filed Oct. 18, 2001, the content of which is incorporated herein by reference.

### FIELD OF THE INVENTION

The present invention is directed to a method for making reconstituted cell lines wherein an entire differentiated cell or the nucleus of a differentiated cell is transplanted into an enucleated differentiated cell. The donor cell or nucleus may be derived from the same species or a different species as the host enucleated cell. The nucleus transfer (NT) unit so generated will exhibit one or more cell surface antigens of the donor nucleus or donor cell. The reconstituted cells can be transplanted into recipient patients for therapeutic purposes, such as the replacement of defective, dead, or dying cells.

### BACKGROUND OF THE INVENTION

Nuclear transfer technology is well known in the art. It is used conventionally as a means to clone mammals and other organisms. In nuclear transfer, a nucleus is taken from a fertilized egg cell or another type of differentiated cell. The nucleus (or even the entire cell) is then transferred into an enucleated oocyte from another individual of the same species. The resulting cellular construct is designated herein as a nuclear transfer (NT) unit. When cultured under appropriate conditions, the NT unit results in new zygote, with the nuclear DNA of the zygote coming from the transferred nucleus.

Nuclear transfer is conventionally performed using donor nuclei (or whole donor cells) and enucleated host cells that are taken from the same species. For example, see U.S. Pat. No. 6,147,276, issued Nov. 14, 2000, to Campbell et al., and U.S. Pat. No. 6,235,970, issued May 22, 2001, to Stice et al. The Campbell et al. patent describes using nuclear transfer to generate embryos for implantation, with the specific aim of generating live offspring having desirable genetic characteristics. The Campbell et al. patent describes a method of generating embryos wherein the nucleus of a quiescent diploid donor cell is transferred into an enucleated recipient oocyte of same species. The NT unit so generated is then activated and cultured to yield an embryo.

Similarly, the Stice et al. patent describes using nuclear transfer to generate cultured inner cell mass (ICM) cell lines. In the method described in the Stice et al. patent, a proliferating, differentiated somatic mammalian donor cell (or a nucleus thereof) is transferred into an enucleated mammalian oocyte of the same species. The resulting NT unit is then activated and cultured to yield ICM cells. The ICM cells are then isolated and cultured separately, to yield a cultured ICM cell line.

Both of these patents, however, describe methods that use donor nuclei and host cells of the same species. Moreover, both of these patents are also limited to methods wherein the host cells are invariably enucleated oocytes, and thus activation procedures are required. The use of host oocytes is due to the emphasis these two patents place on using nuclear transfer as a means to clone livestock, such as cattle and swine, or as a means to generate cultured cell lines of undifferentiated embryonic stem cells. Neither of these two patent address applying nuclear transfer techniques between a differentiated donor cell or nucleus and a differentiated, enucleated host cell, in an effort to generate distinct cell lines having desired functional and immunological characteristics.

In the context of cloning, it is known that enucleated cytoplasm can reprogram a transferred nucleus. In this approach, an enucleated oocyte is fused with a normal diploid nucleus or even an entire, intact diploid cell. This creates a zygote that can develop into an entire animal. See Willadsen (1986) Nature 320:63-65. See also Linder et al. (1979) Exp. Cell Res. 120(l):1-14.

Nuclear transfer has also been conducted using embryonic cancer cells as the donor cells. See Linder et al. (1979)Exp. Cell Res. 120(1):1-14. In this work, F9 cancer cells (a nullipotent embryonal carcinoma cell line) were fused with preliminary enucleated somatic cells from differentiated tissues such as thymus and lens. Pluripotent reconstituted cell lines (i.e., cybrids) were obtained. The reconstituted cell lines or cybrids exhibited a wide spectrum of differentiation, including neural tubes, cartilage, skeletal muscle, ciliated epithelia, etc.

It has been shown that cybrid clones form solid tumors after injection into syngenic mice. See Atsumi et al. (1982) Differentiation, 23(1):83-86. Isolated cells from these tumors differentiated into several cell types in vitro, demonstrating pluripotent properties. Other authors have shown that several clones were isolated after fusing mouse embryonal teratocarcinoma (PCC4) cells with cytoplasts of rat myoblastic cells. Iwakura et al. (1985) Cell, 43:777-791.

The Major Histocompatibility Complex (MHC) is a set of molecules displayed on cell surfaces and is responsible for lymphocyte recognition. The MHC is presented as antigen. The MHC molecules control the immune response through recognition of “self” and “non-self.” Consequently, MHC proteins serve as recognition targets in transplantation rejection. Because there are so many specific serotypes HLA, it is highly unlikely that any two unrelated individuals will have the same HLA pattern.

Every species displays its own type of MHC. Thus, in humans, the MHC is presented as Human Leukocyte Antigens (HLA). In bovines, the MHC is presented as Bovine Leukocyte Antigens (BoLA). Molecules of the HLA and BoLA belong to a group of molecules known as the Imunoglobulin Supergene Family, and are encoded by nucleic DNA. See Fiszer & Kurpisz (1998) Am. J. Reprod. Immunol. 40(3):172-176.

### SUMMARY OF THE INVENTION

The present inventor has found that reconstituted cell lines can be generated by nuclear transfer to yield cultured cell lines that express the Major Histocompatibility Complex (MHC) of the donor cell or donor nucleus. Moreover, the present inventor has found that this can be accomplished using both a differentiated donor cell or donor nucleus transplanted into a differentiated, enucleated host cell. Quite surprisingly, it has been found that even where the host cell is derived from a different species than the donor cell or donor nucleus, the resulting NT unit and its progeny exhibit a pattern of Human Leukocyte Antigens (HLA) that is identical to or remarkably similar to the HLA pattern of the donor cell or donor nucleus.

In this fashion, it is a major goal of the present invention to generate cell lines that can be used to replace defective or dead cells in a mammalian subject requiring functioning cells. Of particular note in the present invention, where the donor nucleus is taken from a human patient, and the host cell is likewise taken from a different human individual, the resulting nuclear transfer unit and its progeny and HLA pattern that matches that of the donor cell type. This is an extraordinary leap forward in transplantation technology.

Specifically, there are so many specific serotypes and patterns of HLA on the surface of mammalian cells that it is extraordinarily unlikely that any two unrelated individuals will have cells that display the same HLA pattern. Using the present method, however, differentiated cells displaying a specific HLA pattern can be generated for any individual. Using a donor cell or a donor nucleus from the individual to be treated, and transferring the same into an enucleated host cell of a healthy individual results in a renewable source of replacement cells to treat any number of diseases and disease conditions.

For example, human leukocytes taken from a patient and transferred into enucleated cytoplasts of healthy hepatocytes yields reconstituted hepatocytes that exhibit the HLA pattern of the donor leukocytes. Thus, it is possible, using the present invention to reprogram patient cells such as leukocytes, embryonic stem cells, or nuclei of fibroblasts, by transferring the cells into any type of enucleated and differentiated cell. The reconstituted cell lines, commonly referred to as cybrids, will not be rejected after transplantation into the individual who donated the transferred nuclei or cells because the cybrids exhibit the same HLA as that of the donor nuclei or donor cells.

Thus, using enucleated cardiac cells, reconstituted cell lines can be generated to treat heart disease. Similarly, pancreatic cells can be used to generate reconstituted cell lines to treat diabetes. Likewise, connective tissue cells can be used to generate reconstituted cell lines for treating osteoarthritis and rheumatoid arthritis.

By this approach, healthy cytoplasm can be used to generate reconstituted cell lines to replace, for example, cells having defective mitochondria. The reconstituted cell lines are fully compatible with the donor of the transferred nuclei or cells. The invention therefore can be used to treat mitochondrial defects in neurodegenerative conditions such as Alzheimer's disease and Parkinson's disease.

The donor cell or donor nucleus can be derived from virtually any mammalian cell type, specifically including (without limitation) blood monocytes, skin fibroblasts, hepatocytes, renal cells, neurons, glial cells, mesothelial cells, and the like. The donor cell or donor nucleus is preferably a human diploid somatic cell, but also may be derived from any non-human mammal. Likewise, the enucleated host cell must be derived from a diploid mammalian source.

The donor cell or donor nucleus may be derived from genetically-modified sources, without limitation. Thus, the donor cell or nucleus may contain chromosomally-integrated or episomal recombinant nucleic acid sequences, the donor cell or nucleus may be an immortalized or an immortalizable cell line, a genetically transformed cell line, and the like.

Thus, a first embodiment of the invention is directed to a method of reconstituting a mammalian cell line. The method comprises first transferring a mammalian, differentiated, diploid donor cell or a donor nucleus of a mammalian, differentiated, diploid cell into a mammalian, differentiated, diploid enucleated host cell, wherein the donor cell or donor nucleus and the host cell are derived from identical species or different species, thereby obtaining a nuclear transfer unit. The nuclear transfer unit may then optionally be activated. Activation is optional and in the preferred embodiment, no activation step is required. The nuclear transfer unit is then incubated to yield a reconstituted mammalian cell line.

The donor cell or donor nucleus and the host cell may be derived from identical mammalian species or from different mammalian species, including Homo sapiens.

A second embodiment of the invention is directed to a method of generating a reconstituted mammalian cell line that displays human leukocyte antigens. Here, the method comprises first transferring a human, differentiated, diploid donor cell or a donor nucleus of a human, differentiated, diploid cell into a non-human mammalian, differentiated, diploid enucleated host cell, thereby obtaining a nuclear transfer unit. The nuclear transfer unit may then optionally be activated. (Again, it is preferred that there be no activation step.) Then, the nuclear transfer unit is incubated to yield a reconstituted mammalian cell line, wherein cells of the reconstituted cell line display human leukocyte antigens.

The non-human enucleated host cell is preferably derived from a primate, bovine, ovine, porcine, or murine species.

Alternatively, the method may proceed as outlined immediately above, however, the derivation of the donor and host cells are reversed. In other words, a non-human, differentiated, diploid donor cell or a donor nucleus of a non-human, differentiated, diploid cell is transferred into a human mammalian, differentiated, diploid enucleated host cell, thereby obtaining a nuclear transfer unit. The NT unit is then incubated as noted previously.

Lastly, the invention further encompasses reconstituted cell lines fabricated according to any of the foregoing methods.

### DETAILED DESCRIPTION

The following definitions are provided for a consistent understanding of the invention described and claimed herein. Words not explicitly defined herein are to be given their accepted meaning in the field of nuclear transplantation.

“Cell Line:” A “cell line” arises from a primary culture at the time of the first successful sub-culture. The term “cell culture” explicitly denotes that cultures from it comprise a lineage of cells originally present in the primary culture. The terms “finite” or “continuous” are used as prefixes if the status of the culture is known.

“Cytoplast:” An intact cytoplasm that remains after the enucleation of a cell.

“Cybrid” or “Reconstituted Cell Line:” These two terms are used synonymously herein and refer to cell lines that result from the fusion of a donor whole cell or nucleus and a cytoplast.

“Karyoplast:” As used herein, “karyoplast” is generally synonymous with “nucleus” as that word is commonly understood. More specifically, however, a “karyoplast” is a cell nucleus obtained from a donor cell via enucleation of the donor cell. Thus, a karyoplast is a cell nucleus that is surrounded by a very narrow rim of cytoplasm and a plasma membrane.

Nuclear transfer techniques for oocytes are conventional and well known to those skilled in the art. See, for example, Campbell et al. (1995) Theriogenology 43:181; Collas et al. (1994) Mol. Report Dev. 38:264-267; Keefer et al. (1994) Biol. Reprod., 50:935-939; Sims et al. (1993) Proc. Natl. Acad. Sci., USA 90:6143-6147; WO 94/26884; WO 94/24274; and WO 90/03432. See also U.S. Pat. Nos. 4,944,384 and 5,057,420, which describe procedures for bovine nuclear transplantation. All of these documents are incorporated herein by reference.

Differentiated cells have a diameter of roughly 15-35 μm, and are much smaller than oocytes, which have a diameter of roughly 100-200 μm. Thus, nuclear transfer techniques for differentiated cells are necessarily slightly different than the techniques used for oocytes. Differentiated mammalian cells are defined herein as those cells which are past the early embryonic stage. More particularly, the differentiated cells are those from at least past the embryonic disc stage (day 10 in bovine embryogenesis). The differentiated cells may be derived from ectoderm, mesoderm or endoderm.

Mammalian diploid cells of any type, including human cells, may be obtained by well known methods. Mammalian cells useful in the present invention include, by way of example, epithelial cells, neural cells, epidermal cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, and other muscle cells, etc. Moreover, the mammalian cells used for nuclear transfer may be obtained from different organs, e.g., skin, lung, pancreas, liver, stomach, intestine, heart, reproductive organs, bladder, kidney, urethra and other urinary organs, etc. These are just examples of suitable donor cells. Suitable donor cells and their nuclei, i.e., cells useful in the subject invention, may be obtained from any cell or organ of the body.

Leukocytes or nuclei of fibroblast cells are an ideal cell type because they can be obtained from developing fetuses and adult animals (including humans) in large quantities. Leukocytes and fibroblasts are differentiated and are generally considered a poor cell type to use in cloning procedures. Fibroblasts are easily propagated in vitro with a rapid doubling time and can be clonally propagated for use in gene targeting procedures.

The present invention is novel because differentiated cell types are used as both the donor and host. The present invention is advantageous because the cells can be easily propagated, genetically modified and selected in vitro.

Suitable mammalian sources for donor and host cells, in addition to humans, include sheep, cows, pigs, horses, rabbits, guinea pigs, mice, hamsters, rats, primates, etc.

Diploid somatic cells can be obtained from any mammalian source, including humans. The primary cell culture from which the biological materials are obtained include, but not limited to, genetically engineered and re-engineered cell lines of patients that may contain chromosomally-integrated or episomal recombinant nucleic acid sequences, immortalized or immortalizable cell lines, differentiated or predetermined cell lines, transformed cell lines, and the like. Separation of karyoplasts or cytoplasts can be performed by high speed centrifugation in a ladder gradient of ficoll in the presence of Cytochalasin B or Cytochalasin D. Other reagents, including but not limited to, other biological or chemical products that disrupt actin filaments in the cytoskeleton may also be used (as has been previously described). See Wigler & Weinstein (1975) Biochem. Biophys. Res. Commun. 63(3):669-674.

Enucleation of cells disposed in a monolayer can be performed as described by Prescott et al. (1972). In brief, a coverslip with adherent cells is incubated at 37° C. for 30 min in the presence of 1.5 mg/ml cytochalasin D (CD; Sigma) and then centrifuged at 15,000 ×g for 40 min at 37° C. in the presence of CD. The cytoplasts are then rinsed with fresh drug-free medium and incubated for 4 h at 37° C. before use.

Cell karyoplasts for fusion can be harvested using above methods to yield the karyoplasts and then isolating the karyoplasts from a ficoll gradient or, in case of an enucleated monolayer, from the bottom of the centrifuge tube. Wigler & Weinstein, supra.

Fusion of cell cytoplasts and nuclei or entire cells can be accomplished using chemical fusion reagents such as polyethylenglycol (PEG), an agglutinin protein like phytohemagglutinin, or concavalin A. If PEG is used, it is preferred that the PEG have a molecular weight range of from 400 and up to 10,000. Fusion may also be accomplished using an inactivated hemagglutinin virus, like Sendai virus. Fusion can also be accomplished via electrofusion.

Fusion of cytoplasts with embryonic stem (ES) cells has been accomplished as described below, and this approach can be utilized in the present invention. The cytoplast band of differentiated cells was recovered and washed with DMEM, then washed with 0.3 M mannitol fusion medium at pH 7.2. A total of 1×10⁷ cytoplasts were mixed with 1×10⁶ R-6G-treated ES cells and fused by electric current delivered as a 20 sec alignment at 50 V alternate current (AC), followed by two 20 μs pulses of 800 V direct current (DC) (2.5 kV/cm) without a post-fusion AC field using a BTX-Genetronics ECM200 electrofusion device (San Diego). After a 2 min recovery time, the cells were plated onto fresh feeders and continued to receive pyruvate and uridine supplementation for 24 h. See Sligh et al. (2000) Proc. Natl. Acad. Sci. USA 97:14461-14466.

Isolation of cybrid colonies can be performed by plating cells at low density on Petri dishes or by aliquoting 1-3 cells per well into 96-wells plates. Growth medium may or may not contain selective agents that allow proliferation or inhibition of specific cell types. Isolated colonies usually appear 3 to 4 weeks after the initial fusion.

The cells obtained and established after fusion (with or without growth selection) are the major subject of the present invention. These established cybrid cell lines express unique MHC patterns; patterns whose expression is driven by the DNA contained within the donor cells or donor nuclei (such as an individual serotype of HLA or BoLA). Thus, cybrid cell lines of the present invention display surface antigens that are histocompatible with the donor individual. These cybrid cells can be used for cell therapy to replace patient cells that are dead, defective, or otherwise require replacement. The pattern of differentiation and/or expression of facultative or constitutive markers of the resulting allogeneic cybrids or reconstituted cells can be modified or changed by adding different biological or chemical agents, induction agents, inhibitors, and/or stabilizers of differentiation, in combination with different cell or tissue culture conditions (depending on the type of cytoplast being used). Further propagation and selection of clones from the reconstituted cell lines yields allogeneic, histocompatible cells for transplantation and tissue regeneration.

Enucleation of the host cell can be accomplished by known methods, such as the method described in U.S. Pat. No. 4,994,384, which is incorporated by reference herein. For example, metaphase II oocytes are either placed in HECM, optionally containing 7.5 μg/ml cytochalasin B, for immediate enucleation, or may be placed in a suitable medium, for example an embryo culture medium such as CR1aa, plus 10% estrus cow serum, and then enucleated later, preferably not more than 24 hours later, and more preferably 16-18 hours later.

Enucleation can also be accomplished microsurgically using a micropipette to remove the nucleus physically. The host cells may then be screened to identify those of which have been successfully enucleated. This screening may be effected by staining the cells with 1 μg/ml 33342 Hoechst dye in HECM, and then viewing the cells under ultraviolet irradiation for less than 10 seconds. The cells that have been successfully enucleated can then be placed in a suitable culture medium, e.g., CR1aa plus 10% serum.

Transferring the donor cell or nucleus into the enucleated host is preferably accomplished by electrofusion. Electrofusion is accomplished by providing a pulse of electricity that is sufficient to cause a transient breakdown of the plasma membrane. This breakdown of the plasma membrane is of very short time duration. Thus, when two adjacent membranes are induced to breakdown, upon reformation of the two membranes, the lipid bilayers will intermingle. Small channels are thus created between the two cells. Due to the thermodynamic instability of such a small opening, the opening enlarges until the two cells (donor and host) become one. See U.S. Pat. No. 4,997,384 for a full discussion of electrofusion. A variety of electrofusion media can be used, including sucrose, mannitol, sorbitol and phosphate buffered solution. Fusion can also be accomplished using Sendai virus as a fusogenic agent.

Also, in some cases (e.g. when working with small donor nuclei) it may be preferable to inject the donor nucleus directly into the host cell, rather than using electrofusion. Such direct injection techniques are disclosed in Collas & Barnes (1994) Mol. Reprod. Dev. 38:264-267.

Preferably, the mammalian cell and oocyte are electrofused in a 300 μm chamber by application of an electrical pulse of 90-120 V for about 15 μsec. After fusion, the resultant fused NT units are then placed in a suitable medium until activation. Typically activation will take place shortly thereafter, typically less than 24 hours later, and preferably about 0-6 hours later.

The NT unit may be activated by known methods. Such methods include, e.g., culturing the NT unit at sub-physiological temperature, in essence by applying a cold, or actually cool temperature shock to the NT unit. This may be most conveniently done by culturing the NT unit at room temperature, which is cold relative to the physiological temperature conditions common to mammals.

Alternatively, activation may be achieved by application of known activation agents. Also, treatments such as electrical and chemical shock may be used to activate NT units after fusion. Activation methods are the subject of U.S. Pat. No. 5,496,720, incorporated herein by reference in its entirety.

Activation may also be accomplished by simultaneously or sequentially:

(i) increasing levels of divalent cations in the NT unit, and

(ii) reducing phosphorylation of cellular proteins in the NT unit.

This will generally be effected by introducing divalent cations into the NT unit cytoplasm, e.g., magnesium, strontium, barium or calcium, e.g., in the form of an ionophore. Other methods of increasing divalent cation levels include the use of electric shock, treatment with ethanol, and treatment with caged chelators.

Phosphorylation may be reduced by known methods, e.g., by adding kinase inhibitors such as serine-threonine kinase inhibitors, e.g., 6-dimethyl-aminopurine, staurosporine, 2-aminopurine, sphingosine, cycloheximide, and combinations thereof.

Alternatively, phosphorylation of cellular proteins may be inhibited by introduction of a phosphatase into the NT unit, e.g., phosphatase 2A and phosphatase 2B.

In one embodiment, NT unit activation is accomplished by exposing the fused NT unit to a TL-HEPES medium containing 5 μM ionomycin and 1 mg/ml BSA, followed by washing in TL-HEPES containing 30 mg/ml BSA within about 24 hours after fusion, and preferably about 0 to 9 hours after fusion.

The activated NT units may then be cultured in a suitable in vitro culture medium. Culture media suitable for culturing and maturation of a huge host of cell types embryos are well known in the art. Examples of known media which may be used for NT unit culture and maintenance, include Ham's F-10+10% fetal calf serum (FCS), Tissue Culture Medium-199 (TCM-199)+10% fetal calf serum, Tyrodes-Albumin-Lactate-Pyruvate (TALP), Dulbecco's Phosphate Buffered Saline (PBS), Eagle's, Hank's, and Whitten's media.

Another maintenance medium is described in U.S. Pat. No. 5,096,822 to Rosenkrans, Jr. et al., which is incorporated herein by reference. This medium is designated CR1. Additionally, a defined quantity of essential and non-essential amino acids may be added to the CR1 medium to yield CR1aa medium. CR1aa medium preferably contains the following components in the following quantities: sodium chloride—114.7 mM; potassium chloride—3.1 mM; sodium bicarbonate—26.2 mM; hemicalcium L-lactate—5 mM; and fatty-acid free BSA—3 mg/ml.

After NT units of the desired size are obtained, the cells are mechanically removed from the zone and are then used. This is preferably accomplished by taking the clump of cells which comprise the NT unit, which typically will contain at least about 50 cells, washing such cells, and plating the cells onto a feeder layer, e.g., irradiated fibroblast cells. The cells are maintained in the feeder layer in a suitable growth medium, e.g., alpha MEM supplemented with 10% FCS and 0.1 mM P-mercaptoethanol (Sigma) and L-glutamine. The growth medium is changed as often as necessary to optimize growth, e.g., about every 2-3 days.

The resultant progeny cells and cell lines, preferably human and/or humanized cells and cell lines, have numerous therapeutic and diagnostic applications. Most notably, the resultant cells may be used for cell transplant therapy.

The subject reconstituted cells may be used to obtain any desired differentiated cell type. Therapeutic uses of these differentiated human cells are unparalleled. For example, human hematopoietic stem cells may be used in medical treatments requiring bone marrow transplantation. Such procedures are used to treat many diseases, e.g., late stage cancers such as ovarian cancer and leukemia, as well as diseases that compromise the immune system, such as AIDS. Hematopoietic cells can be obtained, e.g., by fusing adult somatic cells of a cancer or AIDS patient, e.g., epithelial cells or lymphocytes, with an enucleated epithelial cell or lymphocyte of a healthy individual, and culturing the NT units until hematopoietic cells are obtained. Such hematopoietic cells may be used in the treatment of diseases including cancer and AIDS.

### EXAMPLES

The following Examples are included solely to provide a more complete understanding of the invention described and claimed herein. The Examples do not limit the scope of the claimed invention in any fashion.

### Example 1

### Expression of Human MHC Markers in Differentiated Reconstituted Cell Lines

Human embryonic kidney cells (cell line 293) were enucleated in a ficoll gradient. The ficoll density gradient was created by mixing 5-10% dextran (MW 15,000-20,000) and 10-20% ficoll 400. The cell suspension contained cytohalasin B (final concentration 10 μg/ml). The cell suspension was carefully layered on top of the ficoll and the tube was centrifuged for 75 min and 17,000 ×g. Cytoplasts were collected and washed in Hank's solution and then plated onto 35 mm Petri dishes at a concentration of 3×105 in the presence of heat-inactivated FBS. After 24 hours, non-attached cytoplasts were removed and the purity of the attached cytoplasts was evaluated via phase-contrast microscopy. Cell runs were also examined after fixation with cold glacial acetic acid and methanol (1:10) and staining with Giemsa.

Bovine lymphocytes were isolated from heparinized blood, via ficoll density gradient centrifugation (Hypaque). Bovine lymphocytes isolated from 1 ml of blood were washed with Hank's solution, and carefully layered on top of the attached cytoplasts described in the immediately preceding paragraph. The Petri dishes were centrifuged at 1500 rpm for 10 minutes to provide a tight contact between the enucleated human cytoplasts and the bovine lymphocytes.

Fusion was accomplished chemically. A fusion agent, 50% PEG (MW ˜3000) with 5-10% DMSO was added to the Petri dishes, and the dishes allowed to incubate for 60 to 90 seconds. Fusion was performed in serum-free medium. The fusion agent was then diluted by adding additional serum-free medium. The serum-free medium was then replaced with growth medium and the Petri dishes allowed to incubate for 24 hours.

The fused NT units, formed from human cytoplasts and bovine lymphocytes, proliferated further, thus demonstrating the operability of the present invention. The lymphocytes and cytoplasts that did not fuse, did not proliferate.

After 24 hours, the proliferating NT units were plated at low density to analyze individual clones. The average fusion efficiency for the protocol described in this Example was 20-35% (production of fused cells as compared to total cells).

From two fusion runs, after 24 days of incubation in growth medium, a total of 29 cybrid clones between human cell line 293 and bovine lymphocytes were isolated. These cells exhibited a gross morphology similar to cell line 293. These cell lines proliferated in vitro for over 90 days and were evaluated for marker expression, BoLA (bovine leukocyte antigens), and karyotype. The results of the first fusion run are shown in Table 1; the results from the second fusion run are shown in Table 2:

TABLE 1

| Clones | Serotype BoLA | vm | ck | Karyotype |
|---|---|---|---|---|
| Lymphocytes | A11/w44, w50 | − | − | 2n = 58, XY |
| Cell line 293 | None | + | + | 3n = (62-69), XXX |
| 30A4 | A11/w44, w50 | − | + | 2n = 58, XY |
| 30A8 | A11/w44, w50 | − | − | 2n = 58, XY |
| 30A11 | A11/w44. w50 | + | + | 2n = 58, XY |
| 30B3 | A11/w44, w50 | − | − | 2n = 58, XY |
| 30B6 | A11/w44, w50 | − | − | 2n = 58, XY |
| 30B12 | A11/w44, w50 | + | + | 2n = 58, XY |
| 30B24 | A11/w44, w50 | − | − | 2n = 58, XY |
| 30C1 | N/A | − | − | |
| 30C4 | A11/w44, w50 | − | + | 2n = 58, XY |
| 30C8 | A11/w44, w50 | + | − | 2n = 58, XY |
| 30D9 | N/A | − | + | |
| 30D15 | A11/w44, w50 | + | − | 2n = 58, XY |

In Table 1, the cell line 293 (human embryonic kidney, transformed by adenovirus 5ad5) normally expresses cytokeratin (ck+) and vimentin (vm+), and has a karyotype 3n=62-69, XXX). The bovine lymphocytes used in this Example were taken randomly from slaughterhouse animals. The serotype of the MHC for the bovine lymphocytes used in the first fusion run (and reported in Table 1) was All/w44,w50. Native bovine lymphocytes do not express ck or vm. In similar fashion, native human cells do not express BoLA serotypes.

TABLE 2

| Clones | Serotype BoLA | vm | ck | Karyotype |
|---|---|---|---|---|
| Lymphocytes | w40(A13)/w44, w50 | − | − | 2n = 58, XX |
| Cell line 293 | N/A | + | + | 2n = (62-69), XXX |
| 32A7 | w40(A13)/w44, w50 | − | + | 2n = 58, XX |
| 32A9 | w40(A13)/w44, w50 | − | − | 2n = 58, XX |
| 32A14 | w40(A13)/w44, w50 | − | − | 2n = 58, XXX |
| 32B3 | w40(A13)/w44, w50 | + | + | 2n = 58, XX |
| 32B6 | N/A | − | − | N/A |
| 32B9 | w40(A13)/w44, w50 | + | + | 2n = 58, XX |
| 32B16 | w40(A13)/w44, w50 | − | − | 2n = 58, XX |
| 32C4 | w40(A13), w44, w50 | − | − | 2n = 58, XX |
| 32C10 | N/A | + | + | N/A |
| 32C16 | w40(A13)/w44, w50 | − | + | 2n = 58, XX |
| 32C23 | w40(A13)/w44, w50 | − | − | 2n = 58, XX |
| 32D2 | w40(A13)/w44, w50 | − | − | 2n = 58, XX |
| 32D7 | N/A | + | − | N/A |
| 32D11 | w40(A13)/w44, w50 | + | + | 2n = 58, XX |
| 32D18 | w40(A13)/w44, w50 | − | − | 2n = 58, XX |
| 32F5 | w40(A13)/w44, w50 | − | + | 2n = 58, XX |
| 32F23 | w40(A13)/w44, w50 | + | − | 2n = 58, XX |

In the second fusion run, the serotype of the bovine lymphocytes used was w40(A13)/w44,w50.

As is clearly shown in Tables 1 and 2, the reconstituted cells lines include cybrids that simultaneously express BoLA serotypes and specific cytoplast markers (ck and vm). This example demonstrates that the present method can be used to generate reconstituted cell lines exhibiting HLA markers, even when a non-human donor cell is used. Moreover, this Example shows that the resulting cell lines can be cultured in vivo for at least 90 days.

### Example 2

### Expression of Human Albumin and Alpha-fetoprotein in Clones of Reconstituted Cell Lines Between Fresh Isolated Bovine Hepatocytes and Human Cell Line 293.

Freshly isolated hepatocytes from bovine liver were enucleated in a ficoll density gradient in the same fashion as in Example 1.

Cytoplasts of bovine hepatocytes were collected, washed in Hank's solution and plated onto 35 mm dishes in presence of bovine heat-inactivated serum (cell density 3-5×10⁵ per dish). After 24 hours, non-attached cytoplasts were removed by changing the medium. The purity of the attached cytoplasts was evaluated via phase-contrast microscopy. The cytoplasts were also examined by fixing them in glacial acetic acid and methanol (1:10) and Giemsa staining (not less than 95% pure).

Nuclei of human cell line 293 (human embryonic kidney) were collected as described in Example 1.

Cells of human line 293 contain reduced amounts of HLA A*0301, B*0702, and Cw*0702, because the early region 3 of the human adenovirus types 2 and 5 encodes a 19-kDa glycoprotein that associates with the MHC class 1 antigens in the endoplasmic reticulum. This prevents their maturation and transport to the cell surface. Because of that serotype, cell line 293 was not detectable.

The nuclei of cell line 293 were collected and washed in Hank's solution and placed as suspension of 1 to 1.5 ml volume over the top of enucleated hepatocyte cytoplasts described hereinabove. The dishes were centrifuged at 1500 rpm for 10 min to provide tight contact between the enucleated bovine hepatocyte cytoplasts and the human nuclei.

Fusion was accomplished chemically as described in Example 1. Fusion was performed in serum-free medium, after which the NT units were allowed to incubate in growth medium.

After 24 hours of incubation in growth medium, the fused cytoplasts of bovine hepatocytes with the human cell line 293 yielded proliferating, reconstituted cells.

The reconstituted cells were plated in low density for isolation and analysis of individual clones. The average efficiency of fusion for the protocol described in this Example procedure varied from 15-30% (fused cell vs. total cells).

After 28 days of incubation, a total of 14 reconstituted clonal cell lines were obtained. These cells displayed a gross morphology similar the parent bovine hepatocytes. These 14 clones were carefully selected for further proliferation and analysis.

The cells were then assayed for expression of human alpha-fetoprotein and albumin. Note that cell line 293 does not normally express human alpha-fetoprotein nor albumin. The model karyotype presented by this cell line is 3n=(62-69),XXX.

The bovine hepatocyte cells were taken from a fresh sample of embryonic liver from 6- to 7-month old bovine fetus taken at slaughterhouse. The serotype of MHC of the bovine hepatocytes before fusion was detected as A13/w50 on lymphocytes from the same source. Alpha-fetoprotein was detected by immunophoresis from cell extract against of rabbit polyclonal antibodies.

Albumin was detected by comparable polyacrylamide gel electrophoresis of cell extract and control cell extract. To prevent the appearance of exogenic bovine serum albumin before electrophoresis, reconstituted cell lines were cultured one week on serum-free medium (RPMI 1640).

The results are shown in Table 3:

TABLE 3

| Cell Lines | Serotype | AFP | Alb | Karyotype |
|---|---|---|---|---|
| Hepatocytes | A13/w50 | N/A | + | 2n = 58, XX |
| Cell line 293 | N/A | − | − | 3n = (62-69), XXX |
| 29A3 | − | + | + | 3n = (62-69), XXX |
| 29A14 | − | − | − | 3n = (62-69), XXX |
| 29B7 | − | + | + | 3n = (62-69), XXX |
| 29B9 | N/A | − | − | N/A |
| 29B14 | − | − | − | 3n = (62-69), XXX |
| 29B18 | − | + | + | 3n = (62-69), XXX |
| 29C2 | − | − | − | 3n = (62-69), XXX |
| 29C17 | − | + | + | 3n = (62-69), XXX |
| 29D1 | − | − | − | 3n − (62-69), XXX |
| 29D3 | N/A | + | − | N/A |
| 29D5 | − | + | + | 3n = (62-69), XXX |
| 29D9 | − | − | − | 3n = (62-69), XXX |
| 29F5 | − | + | + | 3n = (62-69), XXX |
| 29F23 | − | − | + | 3n = (62-69), XXX |

As clearly depicted by the results in Table 3, this Example resulted in reconstituted cell lines expressing both alpha-fetoprotein nor albumin. This Example shows that the present method will function using various kinds of diploid cell types as the donor and host cells.

### Example 3

### Allogeneic Reconstituted Cell Lines Isolated After Fusion of Human Blood Monocytes (Lymphocytes) with Cytoplasts of Transformed Human Cell Line 293.

In this Example, cytoplasts of human cell line 293 were obtained as described in the previous Examples. Cytoplasts were examined by contrast-phase microscopy and via staining, as noted previously.

Cells of human line 293 contain reduced amounts of HLA A*0301, B*0702, and Cw*0702, because the early region 3 of the human adenovirus types 2 and 5 encodes a 19-kDa glycoprotein that associates with the MHC class 1 antigens in the endoplasmic reticulum. This prevents their maturation and transport to the cell surface. Because of that serotype, cell line 293 was not detectable.

Monocytes from a whole blood sample were isolated from heparinsed blood in a one-step gradient layer (Hypaque) at a density of 1.077 g/ml. Human lymphocytes isolated from 1 ml of blood were washed with Hank's solution and then mixed with approximately the same volume of isolated human cytoplasts.

The cell ratio for fusion in suspension was 1:2, cytoplasts to blood monocytes. After mixing, the cell suspension tubes were centrifuged at 1200 rpm for 10 min. Fusion was performed in serum-free medium as described previously. Fusion and isolation of the resulting NT units was also performed as described in the previous Examples.

The NT units were rinsed thoroughly, plated in growth medium into plastic dishes, and incubated overnight. Fused human cell cytoplasts with human lymphocytes created cybrid clones adhered to the bottom of the plastic dishes and clearly proliferating. Non-fused monocytes and cytoplasts without nuclei did not proliferate further and were eliminated by changing the medium.

After another 24 hours of incubation, the reconstituted cells were plated at low density for isolation and analysis of individual clones. The average efficiency of fusion in the protocol described in this Example varied from 5-10% (fused cells vs. total cells). After 32 days of proliferation, a total of 4 reconstituted clones between human cell line 293 and human lymphocytes were isolated. These clones had a gross morphology similar to cell line 293.

Cell line 293 normally expresses cytokeratin (ck+) and vimentin (vm+), and has a karyotype of 3n =(62-69),XXX.

Human monocytes marker: Serotype of MHC for human monocytes used in this example was HLA-A2; B7, Bw48; Cwl, C-; DR1, DR4. Karyotype 2n-23,XX.

The results are depicted in Table 4:

TABLE 4

| Cell Lines | Serotype HLA | ck | vm | Karyotype |
|---|---|---|---|---|
| Lymphocytes | A2, Aw24, Bw48, | − | − | 2n = 23, XX |
| | CwI, C—, DR1, DR4 | | | |
| Cell line 293 | Not detected (DNA test | + | + | 3n = (62-69), XXX |
| | A*0301, B*0702, | | | |
| | Cw*0702 | | | |
| 37A2 | A2, Aw24, Bw48, | + | + | 2n = 23, XX |
| | CwI, C—, DR1, DR4 | | | |
| 37A5 | A2, Aw24, Bw48, | − | + | 2n = 23, XX |
| | CwI, C—, DR1, DR4 | | | |
| 37B1 | A2, Aw24, Bw48, | + | + | 2n = 23, XX |
| | CwI, C—, DR1, DR4 | | | |
| 37B3 | A2, Aw24, Bw48, | + | + | 2n = 23, XX |
| | CwI, C—, DR1, DR4 | | | |

This Example shows that allogeneic reconstituted cell lines can be created between diploid human cells. Moreover, this Example shows that these allogeneic reconstituted cell lines express the same serotype patter of HLA as lymphocytes, will also expressing ck and vm.

### Example 4

### Intercellular Transfer of the HeLa Nuclear Genome

Intercellular transfer of HeLa nuclei to fibroblast cytoplasts was achieved by fusion of the fibroblast cytoplasts using polyethylene glycol 1500 (Boehringer Mannheim). Cells in the fusion mixture were cultivated in a selection medium of RPMI 1640, 0.1 mg/ml pyruvate, 50 mg uridine, 15% fetal bovine serum, HAT (Sigma), and 2 mM Oua). On day 10 after fusion, colonies grown in the selection medium were cloned by the cylinder method and the resulting cell lines were then cultivated in standard RPMI 1640.

What is claimed is:

1. A method of reconstituting a mammalian cell line, the method comprising:
(a) transferring a mammalian, differentiated, diploid donor cell or a donor nucleus of a mammalian, differentiated, diploid cell into a mammalian, differentiated, diploid enucleated host cell, wherein the donor cell or donor nucleus and the host cell are derived from identical species or different species, thereby obtaining a nuclear transfer unit; and then
(b) incubating the nuclear transfer unit of step (a) to yield a reconstituted mammalian cell line.

2. The method of claim 1, wherein the donor cell or donor nucleus and the host cell are derived from identical mammalian species.

3. The method of claim 1, wherein the donor cell or donor nucleus and the host cell are derived from different mammalian species.

4. The method of claim 3, wherein the donor cell or donor nucleus and the host cell are derived from different mammalian species having a euploid number of chromosomes.

5. The method of claim 1, wherein the donor cell or donor nucleus is derived from Homo sapiens.

6. The method of claim 1, wherein the host cell is derived from Homo sapiens.

7. The method of claim 1, wherein the donor cell or donor nucleus is derived from Homo sapiens and the host cell is derived from Homo sapiens.

8. The method of claim 1, wherein the donor cell or donor nucleus is transferred into the host cell by a means selected from the group consisting of microinjection means, electrofusion means, chemically-induced means, and biologically-induced means using a virus.

9. The method of claim 1, wherein the donor cell or donor nucleus is genetically modified prior to its being transferred into the host cell.

10. The method of claim 1, wherein the donor cell or donor nucleus and the enucleated host cell are non-tumorigenic.

11. A reconstituted mammalian cell line fabricated according to the method of claim 1.

12. A method of generating a reconstituted mammalian cell line that displays human leukocyte antigens, the method comprising:
(a) transferring a human, differentiated, diploid donor cell or a donor nucleus of a human, differentiated, diploid cell into a non-human mammalian, differentiated, diploid enucleated host cell, thereby obtaining a nuclear transfer unit; and then
(b) incubating the nuclear transfer unit of step (a) to yield a reconstituted mammalian cell line, wherein cells of the reconstituted cell line display human leukocyte antigens.

13. The method of claim 12, wherein in step (a), the non-human enucleated host cell is derived from a primate, bovine, ovine, porcine, or murine species.

14. The method of claim 12, wherein in step (a), the donor cell or donor nucleus is transferred into the host cell by a means selected from the group consisting of microinjection means, electrofusion means, chemically-induced means, and biologically-induced means using a virus.

15. The method of claim 12, wherein the donor cell or donor nucleus is genetically modified prior to its being transferred into the host cell.

16. A reconstituted mammalian cell line fabricated according to the method of claim 12.

17. A method of generating a reconstituted mammalian cell line that displays human leukocyte antigens, the method comprising:
(a) transferring a non-human, differentiated, diploid donor cell or a donor nucleus of a non-human, differentiated, diploid cell into a human mammalian, differentiated, diploid enucleated host cell, thereby obtaining a nuclear transfer unit; and then
(b) incubating the nuclear transfer unit of step (a) to yield a reconstituted mammalian cell line, wherein cells of the reconstituted cell line display human leukocyte antigens.

18. The method of claim 17, wherein in step (a), the non-human donor cell is derived from a primate, bovine, ovine, porcine, or murine species.

19. The method of claim 17, wherein in step (a), the donor cell or donor nucleus is transferred into the host cell by a means selected from the group consisting of microinjection or electrofusion.

20. The method of claim 17, wherein the donor cell or donor nucleus is genetically modified prior to its being transferred into the host cell.

21. A reconstituted mammalian cell line fabricated according to the method of claim 17.
